(19) United States
(12) Patent Application Publication
(10) Pub. No.: US 2026/0128162 A1
(43) Pub. Date: May 7, 2026

(54) SYSTEMS AND METHODS FOR COMPARING MEDICAL IMAGES

(71) Applicant: DePuy Ireland Unlimited Company, Ringaskiddy (IE)

(72) Inventors: Bethany Grant, Leeds (GB); Christopher Hunt, Leeds (GB); Jackson R. Heavener, Warsaw, IN (US)

(21) Appl. No.: 19/332,722

(22) Filed: Sep. 18, 2025

Related U.S. Application Data
(60) Provisional application No. 63/715,204, filed on Nov. 1, 2024.

(51) Int. Cl. G16H 30/40; G06T 7/00; G16H 20/40; G16H 30/20; G16H 50/20

(52) U.S. Cl. CPC G16H 30/40; G06T 7/0012; G16H 20/40; G16H 30/20; G16H 50/20; G06T 2207/10081; G06T 2207/10121; G06T 2207/20101; G06T 2207/30012; G06T 2207/30052

(57) ABSTRACT

A computer-implemented method for comparing medical images may include obtaining image data produced by multiple imaging devices indicative of anatomical features associated with an arthroplasty procedure, preprocessing the image data, including projecting three dimensional image data produced by a first imaging device of the multiple imaging devices to a plane represented by two dimensional image data produced by a second imaging device of the multiple imaging devices, identifying one or more landmarks associated with the anatomical features for comparison between images in the image data, comparing the identified one or more landmarks across the images to determine whether the images represent different individuals, and producing a notification indicative of a result of the determination of whether the images represent different individuals.

This application claims the benefit of U.S. Provisional Patent Application Ser. No. 63/715,204, filed Nov. 1, 2024, the entirety of which is incorporated herein by reference.

### TECHNICAL FIELD

The present disclosure relates to the analysis of medical images and more specifically to determining whether a set of medical images represent the same individual.

### BACKGROUND

For medical procedures, such as surgeries to restore the function of a joint, or other orthopedic procedures, a medical practitioner may review anatomical images of a patient produced by one or more imaging devices. Those images may be utilized for preoperative, intraoperative, and/or postoperative purposes, such as for selecting, configuring, and fitting an implant (e.g., an artificial joint or component of an artificial joint) and/or subsequently analyzing the performance of the implant in the patient's body. Without robust analysis and planning in these phases, complications may arise during or after the operation. For example, in the case of a total hip arthroplasty, the complications may include leg length discrepancies, impingement, movement limitations, discomfort, component dislocation, and/or premature component failure.

Some systems, such as VELYS Hip Navigation, from DePuy Synthes, may perform an analysis of medical images for such preoperative, intraoperative, or postoperative purposes based on a collection of medical images selected by a user (e.g., a medical practitioner). Those images may be selected from a larger set of images that may have been produced by multiple imaging devices and that may include medical images for multiple different patients. As such, to ensure that the analysis system receives the correct images, a medical practitioner may expend considerable time verifying that a collection of images to be analyzed do indeed represent the same individual.

Presently disclosed embodiments provide automated analysis of medical images to determine whether the medical images all represent the same individual.

### SUMMARY

According to one aspect of the present disclosure, a computer-implemented method for comparing medical images may comprise obtaining (with a compute device) image data produced by multiple imaging devices indicative of anatomical features associated with an arthroplasty procedure, preprocessing (with the compute device) the image data, including projecting three dimensional image data produced by a first imaging device of the multiple imaging devices to a plane represented by two dimensional image data produced by a second imaging device of the multiple imaging devices, identifying (with the compute device) one or more landmarks associated with the anatomical features for comparison between images in the image data, comparing (with the compute device) the identified one or more landmarks across the images to determine whether the images represent different individuals, and producing (with the compute device) a notification indicative of a result of the determination of whether the images represent different individuals.

In some embodiments, obtaining image data produced by multiple imaging devices may comprise obtaining two dimensional X-ray image data produced by a fluoroscope and three dimensional computed tomography image data produced by a computed tomography imaging device.

In some embodiments, obtaining image data may comprise obtaining preoperative, intraoperative, or postoperative image data of one or more joints.

In some embodiments, the one or more joints may comprise a joint of a pelvis or a spine.

In some embodiments, projecting the three dimensional image data to a plane represented by the two dimensional image data may comprise projecting the three dimensional image data to a sagittal plane, a coronal plane, a transverse plan, or an oblique plane represented in the two dimensional image data.

In some embodiments, preprocessing the image data may comprise establishing a common scale among the images in the image data.

In some embodiments, establishing a common scale may comprise establishing a common pixel density among the images in the image data.

In some embodiments, identifying one or more landmarks to compare may comprise identifying one or more landmarks based on a predefined set of landmarks to be identified.

In some embodiments, identifying one or more landmarks to compare may comprise identifying one or more landmarks to compare based on a degree to which each landmark in a set of possible landmarks is represented in the images.

In some embodiments, identifying one or more landmarks to compare based on a degree to which each landmark is represented in the images may comprise identifying the one or more landmarks based on one or more of a clarity of each landmark in each of the images or an anatomical plane represented in each of the images.

In some embodiments, identifying one or more landmarks may comprise identifying a sacral endplate.

In some embodiments, the method may further comprise determining, with the compute device, a distance between opposite edges of the sacral endplate.

In some embodiments, identifying one or more landmarks may comprise identifying an anterior superior iliac spine (ASIS) and a pubic symphysis.

In some embodiments, the method may further comprise determining, by the compute device, a distance between the ASIS and the pubic symphysis.

In some embodiments, identifying one or more landmarks may comprise identifying a femoral head center and a sacral slope midpoint.

In some embodiments, the method may further comprise determining, by the compute device, a distance between the femoral head center and the sacral slope midpoint.

In some embodiments, comparing the identified one or more landmarks across the images may comprise determining differences in locations of the landmarks across the images.

In some embodiments, comparing the identified one or more landmarks across the images may comprise determining differences in distances between the landmarks across the images.

In some embodiments, comparing the identified one or more landmarks across the images may comprise determining whether differences in the landmarks across the images satisfy a similarity threshold.

In some embodiments, producing a notification may comprise producing a notification indicative of differences in the one or more landmarks across the images in response to a determination that the images represent different individuals.

According to another aspect, the present disclosure includes embodiments of machine-readable storage media comprising a plurality of instructions stored thereon that, in response to being executed by a compute device, cause the compute device to perform the steps of any of the methods described herein.

According to still another aspect, the present disclosure includes embodiments of systems for comparing medical images comprising circuitry configured to perform the steps of any of the methods described herein.

### BRIEF DESCRIPTION OF THE DRAWINGS

The detailed description particularly refers to the following figures, in which:

FIG. 1 is a diagram of at least one embodiment of a system for comparing medical images to determine whether the medical images represent the same individual;

FIG. 2 is a diagram of at least one embodiment of a compute device that may be used in the system of FIG. 1;

FIGS. 3A-3H are a flowchart of at least one embodiment of a method that may be performed by the system of FIG. 1 for comparing medical images to determine whether the same individual is represented in the medical images; and

FIG. 4 shows a set of medical images and landmarks within the medical images that may be compared by the system of FIG. 1 to determine whether the medical images represent the same individual.

### DETAILED DESCRIPTION

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

Disclosed embodiments facilitate analysis of images of anatomical features (medical images) to determine whether the images represent the same individual. In some embodiments, the systems and methods may be utilized in connection with orthopedic procedures, such as arthroplasty. In general, arthroplasty refers to a surgical procedure in which the function (e.g., range of motion and stability) of a damaged joint is restored. In performing an arthroplasty, all or a portion of a joint may be replaced with a prosthetic version thereof. For example, in a total hip arthroplasty (THA), both the acetabulum (i.e., a socket of a hipbone) and the head of the corresponding femur is replaced with artificial components designed to restore the function of that joint. By contrast, hip hemi-arthroplasty involves the replacement of just one of those components of the hip joint (i.e., the femoral head).

Due to the idiosyncrasies in the anatomy of each patient, in a preoperative phase of an arthroplasty, a medical practitioner typically analyzes images representing features of the joint and surrounding area select to a suitable prosthetic. A successful prosthetic femoral head, for example, must fit within the corresponding acetabulum and enable movement of the joint through a defined range of motion, and the neck between the artificial femoral head and stem should be sized to ensure that the length of the affected leg matches the length of the other leg. Similarly, in the intraoperative phase (e.g., during the surgery), the medical practitioner may utilize images of the anatomy to ensure that the prosthetic is implanted in the intended location and in the postoperative phase, the medical practitioner may refer to images of the patient's anatomy to determine whether the prosthetic is operating as intended (e.g., enabling the expected range of motion). With recent advances in software and imaging, all or a portion of the above functions may be offloaded to a compute device. However, the analysis may be adversely affected if the supplied images do not all represent the same person. That is, if certain images indicate different positions of anatomical features that may not be readily apparent to the human eye, the precision with which a suitable prosthetics can be selected may be reduced, and the surgeon may need to utilize more trial prosthetics before identifying the best fit. Similarly, any analysis of the placement and functionality of the prosthetic may be compromised if the images do not represent the same individual, and may lead to negative outcomes including an unintended lateral offset of the affected leg from the hip, a different-than-expected range of motion, and/or displacement of the femoral head from the acetabulum. Further, while utilizing multiple imaging devices may provide an abundance of information about the anatomical area of interest, differences in the format and type of data produced by each of the imaging devices may introduce further complexities in determining whether a set of images all represent the same individual. For example, some imaging devices may produce two dimensional data representing anatomy in two spatial dimensions (e.g., an X-ray device, such as a fluoroscope, or a 2D ultrasound device) while other imaging devices may produce three dimensional image data representing anatomy in three spatial dimensions (e.g., a computed tomography (CT) imaging device, a magnetic resonance imaging (MRI) device, or a 3D ultrasound device).

As described in more detail herein, disclosed embodiments facilitate automated determination of whether a set of supplied medical images represent the same individual, even when the medical images are produced from different imaging devices (e.g., an X-ray imaging device, such as a fluoroscope, a CT imaging device, an MRI device, an ultrasound device). In some embodiments, a compute device may perform this automated detection for every set of multiple images before they are used by the compute device for analysis. In other embodiments, the automated detection of whether a set of supplied medical images represent the same individual may be initiated by the presence of one or more specific triggering conditions, such as one or more Digital Imaging and Communications in Medicine (DICOM) tags (e.g., patient name) associated with the images of the set not matching one another.

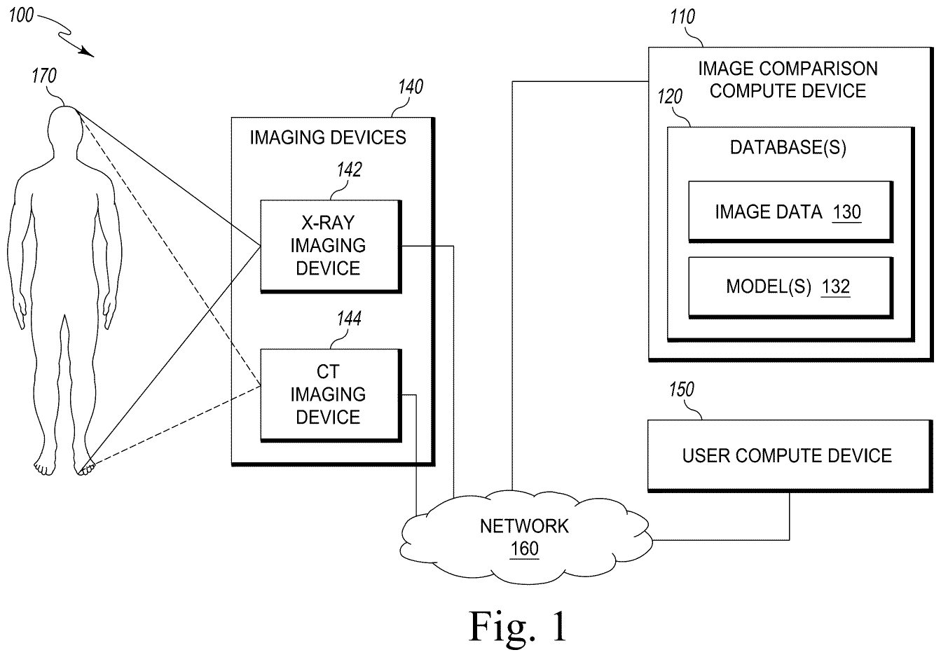

FIG. 1 is a diagram of a system 100 for comparing medical images to determine whether the medical images represent the same individual. In the illustrative embodiment, the system 100 includes an image comparison compute device 110 communicatively connected to a set of imaging devices 140 and a user compute device 150 via a network 160. The imaging devices 140, in the illustrative embodiment, include an X-ray imaging device 142 and a computed tomography (CT) imaging device 144. In the illustrative embodiment, the X-ray imaging device 142 (e.g., a fluoroscope) produces two dimensional image data (e.g., images having two spatial dimensions) and the CT imaging device 144 produces three dimensional image data (e.g., having three spatial dimensions). It will be appreciated that, in other embodiments, the imaging devices 140 may include additional and/or different imaging devices, such as MRI devices and/or ultrasound devices.

In operation, the imaging devices 140 produce medical images (e.g., images representing the anatomy) of a patient 170. The image comparison compute device 110, in operation, may access one or more databases 120 that includes image data 130. In the illustrative embodiment, the image data 130 includes medical images produced by the imaging devices 140. In some embodiments, the image data 130 may be communicated from the imaging devices 140 to the image comparison compute device 110 (e.g., via the network 160). In other embodiments, the image data 130 may be stored by an intermediary device (e.g., in a data storage of a user compute device 150 communicative connected to the imaging devices 140) and transmitted to the image comparison compute device 110 by that intermediary device (e.g., the user compute device 150).

In operation, the image comparison compute device 110 compares a set of medical images (e.g., selected by a user (e.g., of the user compute device 150) from a collection of medical images in the image data 130) and determines whether the medical images represent the same individual (e.g., the patient 170). In doing so, the image comparison compute device 110, in the illustrative embodiment, identifies one or more landmarks (e.g., one or more selected anatomical features from a set of anatomical features) within the medical images and compares those landmarks across the medical images. In doing so, the image comparison compute device 110 may compare the relative locations of the landmarks across the medical images. For example, as described in more detail herein, the image comparison compute device 110 may identify the location of the sacral endplate of S1, determine the distance between opposite edges (e.g., an anterior edge and a posterior edge) of the sacral endplate in each medical image, and compare that distance across the medical images. Similarly, the image comparison compute device 110 may identify the anterior superior iliac spine (ASIS) and the pubic symphysis, determine the distance between them in each of the medical images, and compare the determined distances across the medical images. As yet another example, the image comparison compute device 110 may identify the center of a femoral head and the sacral slope midpoint, determine the distance between them in each medical image, and compare the determined distances across the medical images. In some embodiments, as described in more detail herein, the image comparison compute device 110 may utilize one or more machine learning models 132 to identify landmarks and/or compare the locations of the landmarks across the medical images.

In the event that differences in the locations of the landmarks do not satisfy a defined threshold (e.g., differences in the determined different distances do not satisfy the threshold), the image comparison compute device 110, in the illustrative embodiment, determines that the images do not represent the same individual and produces a notification of that determination. By doing so, the system 100 reduces the likelihood that medical images representing different individuals are inadvertently used for a medical procedure. As such, the system 100 enables more accurate and precise determinations as to the selection and configuration of medical devices (e.g., implants) for use in a patient, as well as the installation and the postoperative analysis of the medical devices.

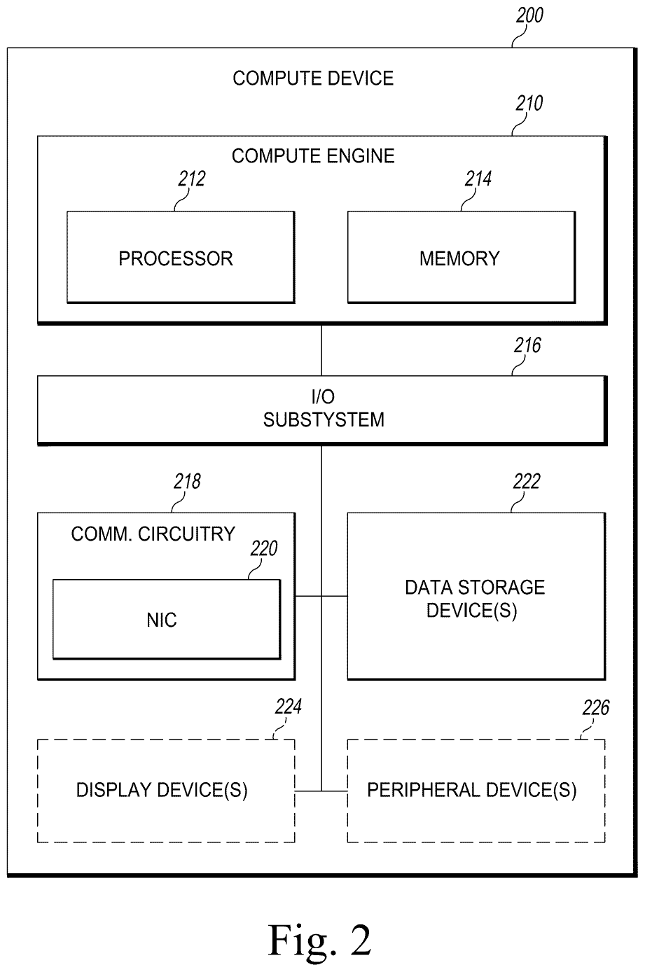

Referring now to FIG. 2, an illustrative embodiment of a compute device 200, representative of each of the devices 110, 140, 142, 144, 150, includes a compute engine 210, an input/output (I/O) subsystem 216, communication circuitry 218, and one or more data storage devices 222. In some embodiments, the compute device 200 may include one or more display devices 224 and/or one or more peripheral devices 226 (e.g., a mouse, a physical keyboard, etc.). In some embodiments, one or more of the illustrative components may be incorporated in, or otherwise form a portion of, another component. The compute engine 210 may be embodied as any type of device or collection of devices capable of performing various compute functions. In some embodiments, the compute engine 210 may be embodied as a single device such as an integrated circuit, an embedded system, a field-programmable gate array (FPGA), a system-on-a-chip (SOC), or other integrated system or device. Additionally, in the illustrative embodiment, the compute engine 210 includes or is embodied as at least one processor 212 and a memory 214. The processor 212 may be embodied as any type of processor capable of performing the functions described herein. For example, the processor 212 may be embodied as a single or multi-core processor(s), a microcontroller, or other processor or processing/controlling circuit. In some embodiments, the processor 212 may be embodied as, include, or be coupled to an FPGA, an application specific integrated circuit (ASIC), one or more graphics processing units (GPUs), neural processing units (NPUs), and/or floating point units (FPUs), reconfigurable hardware or hardware circuitry, or other specialized hardware to facilitate performance of the functions described herein.

In embodiments, the processor 212 is capable of receiving, e.g., from the memory 214 or via the I/O subsystem 216, a set of instructions which when executed by the processor 212 cause the compute device 200 to perform one or more operations described herein. In embodiments, the processor 212 is further capable of receiving, e.g., from the memory 214 or via the I/O subsystem 216, one or more signals from external sources, e.g., from the peripheral devices 226 or via the communication circuitry 218 from an external compute device, external source, or external network. As one will appreciate, a signal may contain encoded instructions and/or information. In embodiments, once received, such a signal may first be stored, e.g., in the memory 214 or in the data storage device(s) 222, thereby allowing for a time delay in the receipt by the processor 212 before the processor 212 operates on a received signal. Likewise, the processor 212 may generate one or more output signals, which may be transmitted to an external device, e.g., an external memory or an external compute engine via the communication circuitry 218 or, e.g., to one or more display devices 224. In some embodiments, a signal may be subjected to a time shift in order to delay the signal. For example, a signal may be stored on one or more storage devices 222 to allow for a time shift prior to transmitting the signal to an external device. One will appreciate that the form of a particular signal will be determined by the particular encoding a signal is subject to at any point in its transmission (e.g., a signal stored will have a different encoding than a signal in transit, or, e.g., an analog signal will differ in form from a digital version of the signal prior to an analog-to-digital (A/D) conversion).

The main memory 214 may be embodied as any type of volatile (e.g., dynamic random access memory (DRAM), etc.) or non-volatile memory or data storage capable of performing the functions described herein. Volatile memory may be a storage medium that requires power to maintain the state of data stored by the medium. In some embodiments, all or a portion of the main memory 214 may be integrated into the processor 212. In operation, the main memory 214 may store various software and data used during operation such as image data, models, applications, libraries, and drivers.

The compute engine 210 is communicatively coupled to other components of the compute device 200 via the I/O subsystem 216, which may be embodied as circuitry and/or components to facilitate input/output operations with the compute engine 210 (e.g., with the processor 212 and the main memory 214) and other components of the compute device 200. For example, the I/O subsystem 216 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O subsystem 216 may form a portion of a system-on-a-chip (SoC) and be incorporated, along with one or more of the processor 212, the main memory 214, and other components of the compute device 200, into the compute engine 210.

The communication circuitry 218 may be embodied as any communication circuit, device, or collection thereof, capable of enabling communications over a network between the compute device 200 and another device (e.g., a device 110, 140, 142, 144, 150, etc.). The communication circuitry 218 may be configured to use any one or more communication technology (e.g., wired or wireless communications) and associated protocols (e.g., Ethernet, Wi-Fi®, WiMAX, Bluetooth®, etc.) to effect such communication.

The illustrative communication circuitry 218 includes a network interface controller (NIC) 220. The NIC 220 may be embodied as one or more add-in-boards, daughter cards, network interface cards, controller chips, chipsets, or other devices that may be used by the compute device 200 to connect with another device (e.g., a device 110, 140, 142, 144, 150, etc.). In some embodiments, the NIC 220 may be embodied as part of a system-on-a-chip (SoC) that includes one or more processors, or included on a multichip package that also contains one or more processors. In some embodiments, the NIC 220 may include a local processor (not shown) and/or a local memory (not shown) that are both local to the NIC 220. Additionally or alternatively, in such embodiments, the local memory of the NIC 220 may be integrated into one or more components of the compute device 200 at the board level, socket level, chip level, and/or other levels.

Each data storage device 222, may be embodied as any type of device configured for short-term or long-term storage of data such as, for example, memory devices and circuits, memory cards, hard disk drives, solid-state drives, or other data storage device. Each data storage device 222 may include a system partition that stores data and firmware code for the data storage device 222 and one or more operating system partitions that store data files and executables for operating systems.

Each display device 224 may be embodied as any device or circuitry (e.g., a liquid crystal display (LCD), a light emitting diode (LED) display, a cathode ray tube (CRT) display, etc.) configured to display visual information (e.g., text, graphics, etc.) to a user. In some embodiments, a display device 224 may be embodied as a touch screen (e.g., a screen incorporating resistive touchscreen sensors, capacitive touchscreen sensors, surface acoustic wave (SAW) touchscreen sensors, infrared touchscreen sensors, optical imaging touchscreen sensors, acoustic touchscreen sensors, and/or other type of touchscreen sensors) to detect selections of on-screen user interface elements or gestures from a user.

In the illustrative embodiment, the components of the compute device 200 are housed in a single unit. However, in other embodiments, the components may be in separate housings. It should be appreciated that while the compute device 200 is representative of the devices 110, 140, 142, 144, 150, any of the devices 110, 140, 142, 144, 150 may include other components, sub-components, and devices (e.g., radiation sources and radiation detectors) that are not discussed above in reference to the compute device 200 and not discussed herein for clarity of the description.

In the illustrative embodiment, the devices 110, 140, 142, 144, 150 are in communication via a network 160, which may be embodied as any type of wired or wireless communication network, including global networks (e.g., the internet), wide area networks (WANs), local area networks (LANs), digital subscriber line (DSL) networks, cable networks (e.g., coaxial networks, fiber networks, etc.), cellular networks (e.g., Global System for Mobile Communications (GSM), Long Term Evolution (LTE), Worldwide Interoperability for Microwave Access (WiMAX), 3G, 4G, 5G, etc.), a radio area network (RAN), or any combination thereof.

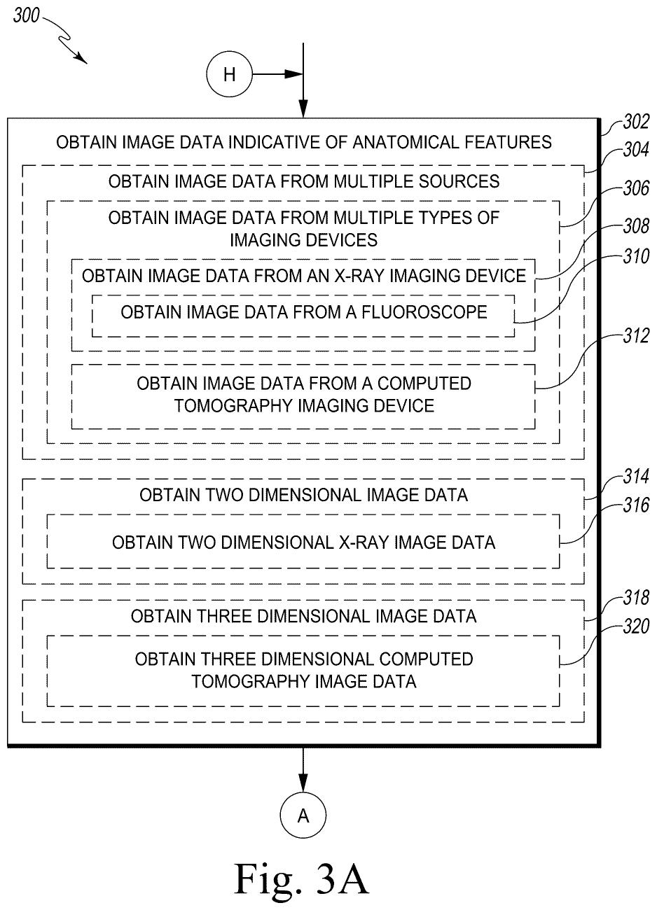

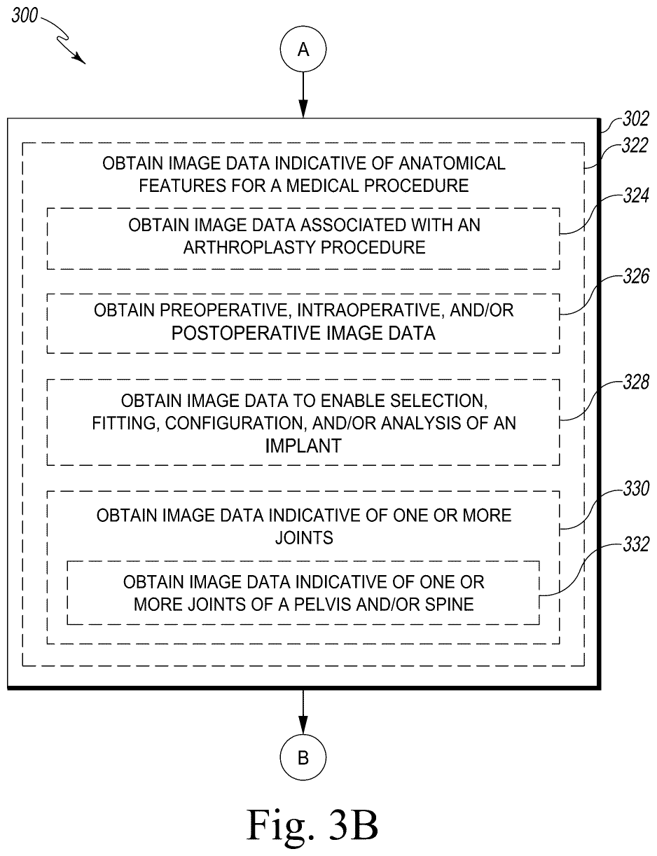

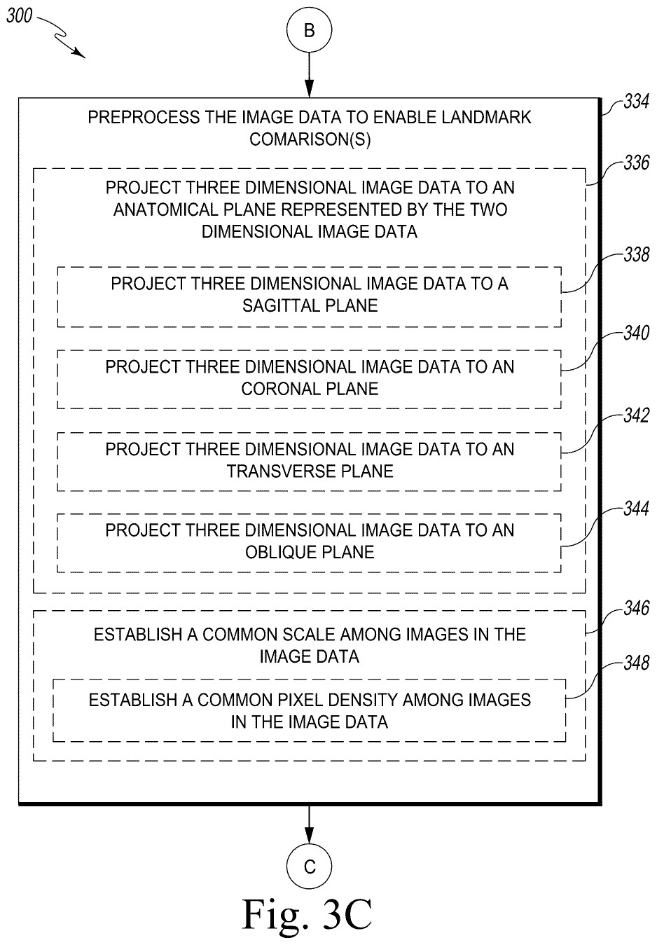

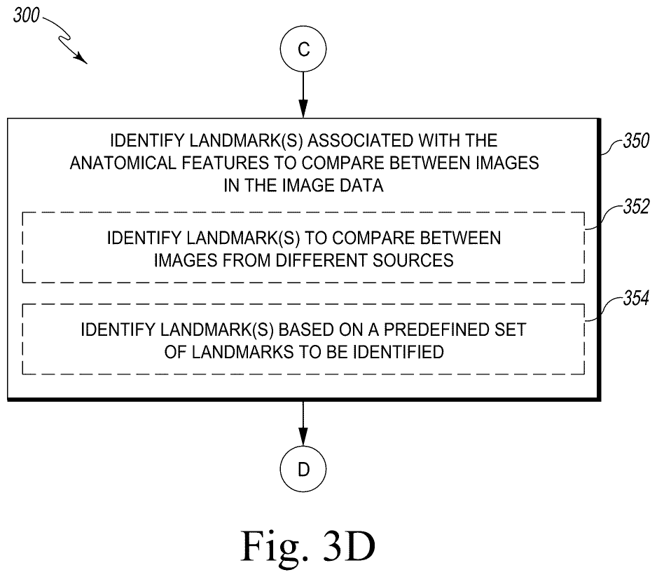

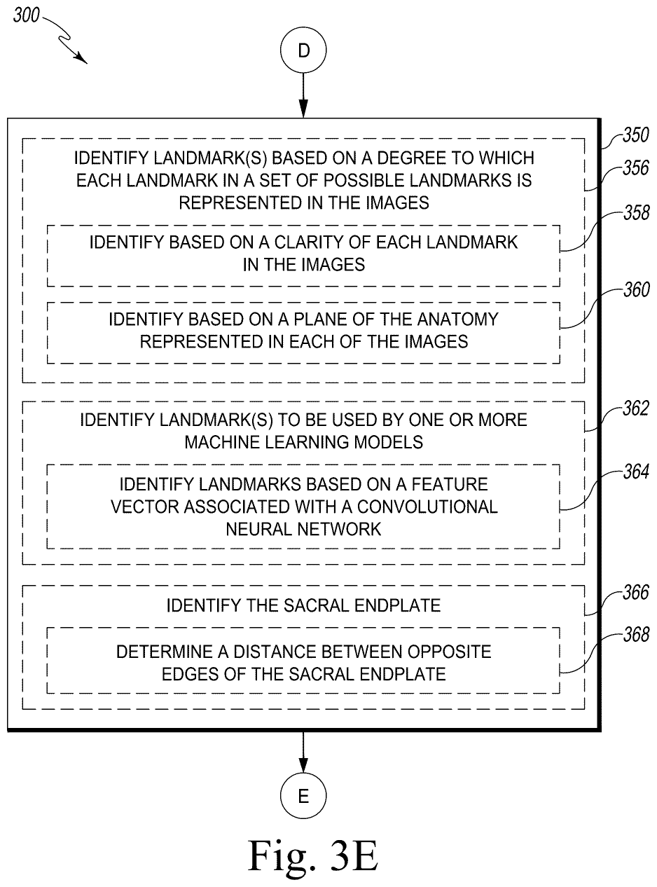

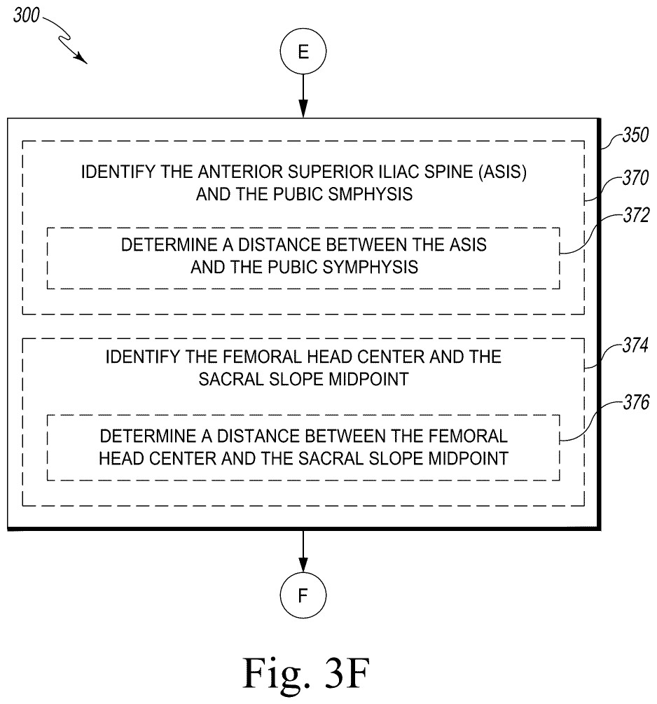

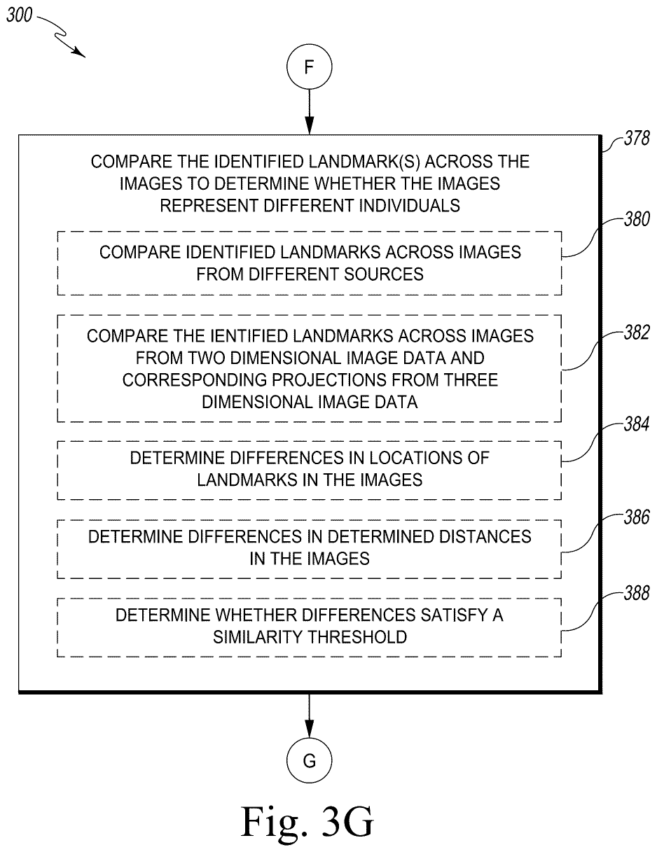

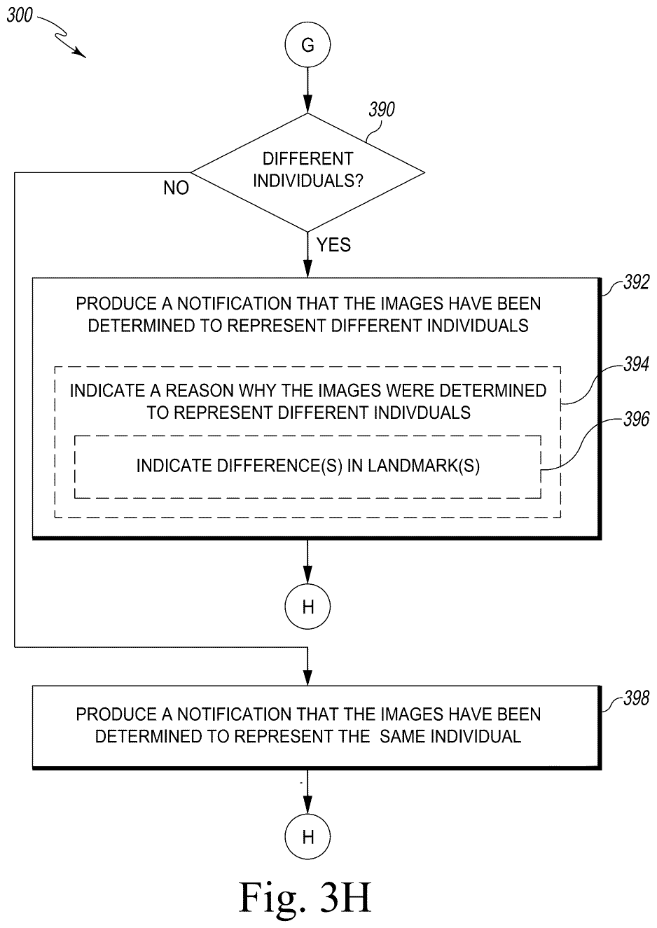

Referring now to FIGS. 3A-3H, in operation, the system 100 (e.g., the image comparison compute device 110) may perform a method 300 for comparing medical images to determine whether the same individual is represented in the medical images. The method 300, in the illustrative embodiment, begins in block 302 (which spans FIGS. 3A-3B), in which the image comparison compute device 110 obtains image data (e.g., the image data 130) indicative of anatomical features. In doing so, as indicated in block 304, the image comparison compute device 110, in the illustrative embodiment, obtains image data from multiple sources. In at least some embodiments, in obtaining image data from multiple sources, the image comparison compute device 110 obtains image data from multiple types of imaging devices 140, as indicated in block 306. For example, as indicated in block 308, the image comparison compute device 110 may obtain image data (e.g., medical image(s)) from an X-ray imaging device (e.g., the X-ray imaging device 142). In doing so, the image comparison compute device 110 may obtain image data from a fluoroscope (e.g., the X-ray imaging device 142 may be embodied as a fluoroscope), as indicated in block 310. The image comparison compute device 110 may also obtain image data from a computed tomography (CT) imaging device (e.g., the CT imaging device 144), as indicated in block 312. In some embodiments of block 306, the compute device 110 may additionally or alternatively obtain image data from one or more other imaging devices 140, such as an MRI device and/or an ultrasound device. In some embodiments, the imaging devices 140 may send the image data 130 (e.g., medical images) directly to the image comparison compute device 110 while in other embodiments, the image comparison compute device 110 may obtain the image data 130 from an intermediary device (e.g., a user compute device 150 that may upload or otherwise provide the images to the image comparison compute device 110).

In obtaining the image data 130, the image comparison compute device 110 may obtain two dimensional image data (e.g., medical image(s) with two spatial dimensions) of the anatomy of a patient (e.g., the patient 170), as indicated in block 314. In some embodiments, the image comparison compute device 110 may obtain two dimensional X-ray data (e.g., data produced by emitting X-ray radiation from a radiation source through at least a portion of the body of the patient 170 and detecting an absorption (i.e., attenuation) of the X-ray radiation by a corresponding radiation detector along two spatial dimensions), as indicated in block 316. Further, the image comparison compute device 110 may obtain three dimensional image data, as indicated in block 318. In some embodiments, the image comparison compute device 110 may obtain three dimensional computed tomography data (e.g., a set of two dimensional images created by detecting the absorption, by a body, of penetrating radiation across a range of angles and that have been computationally combined to form a three dimensional representation of the inside of the body), as indicated in block 320.

Moving to FIG. 3B, the image comparison compute device 110, in the illustrative embodiment, obtains image data 130 that is indicative of anatomical features (e.g., bodily structures, attributes, etc. of an organism) for use in a medical procedure, as indicated in block 322. For example, in some embodiments, the image comparison compute device 110 may obtain image data associated with an arthroplasty procedure (e.g., a partial or total joint replacement), as indicated in block 324. The image comparison compute device 110 may obtain image data for preoperative (e.g., before an operation), intraoperative (e.g., during the operation), and/or postoperative (e.g., after the operation) phases of the medical procedure, as indicated in block 326. Further, the image comparison compute device 110 may obtain image data that was produced to enable selection, fitting, configuration, and/or analysis of an implant (e.g., a prosthetic joint or component of a joint), and as such, may represent anatomical features associated with (e.g., within a predefined distance of) the site of the implant, as indicated in block 328. As indicated in block 330, the image comparison compute device 110 may obtain image data that is indicative of one or more joints. For example, as indicated in block 332, the image comparison compute device 110 may obtain image data indicative of one or more joints of a pelvis and/or spine. In other embodiments, the image comparison compute device 110 may obtain image data indicative of one or more other joints (e.g., knee, ankle, neck, shoulder, etc.) associated with a medical procedure (e.g., arthroplasty).

Continuing the method 300 and referring now to FIG. 3C, the image comparison compute device 110 may preprocess the image data (from block 302) to enable landmark comparisons (e.g., comparison of landmarks across the medical images in the image data 130), as indicated in block 334. In doing so, as indicated in block 336, the image comparison compute device 110 may project three dimensional image data (e.g., from the CT imaging device 144) to an anatomical plane represented by corresponding two dimensional image data (e.g., a cross sectional image produced by the X-ray imaging device 142). That is, the image comparison compute device 110 may perform a planar projection to linearly map each point in three dimensional space (e.g., in the three dimensional image data) to a point on a two dimensional projection plane, such that the resulting point on the projection plane is collinear with the three dimensional point and the center of projection. In doing so, the image comparison compute device 110 may project the three dimensional image data to a sagittal plane (e.g., a vertical plane that extends from front to back through the center of the body) as indicated in block 338. The image comparison compute device 110 may additionally or alternatively project the three dimensional image data to a coronal plane (e.g., a vertical plane that passes through the body longitudinally at a right angle to the sagittal plane and divides the body into a front (anterior) section and a back (posterior) section), as indicated in block 340. The image comparison compute device 110 may project three dimensional image data to a transverse plane (e.g., a horizontal plane that is transverse to the sagittal and coronal planes), as indicated in block 342. In some embodiments, the image comparison compute device 110 may project three dimensional image data to an oblique plane (e.g., a plane that is not parallel or orthogonal to any of the sagittal, coronal, or transverse planes), as indicated in block 344.

Additionally or alternatively, in preprocessing the image data, the image comparison compute device 110 may establish a common scale among images (e.g., medical images) in the image data 130, as indicated in block 346. In doing so, as indicated in block 348, the image comparison compute device 110 may establish a common pixel density among the images in the image data 130. The image comparison compute device 110 may do so by determining the present pixel density of each image, such as by identifying an object of known size (e.g., a metal calibration sphere having a defined diameter) in each of the images, determining the number of pixels representing the object, and dividing the number of pixels by the known size to determine the pixel density. In other embodiments, the pixel density may be defined in metadata associated with each image or in another data set. After determining the present pixel density of each image, the image comparison compute device 110 may adjust the scale by selectively resampling (e.g., through nearest-neighbor interpolation, bilinear interpolation, or bicubic interpolation) each image to have a target pixel density (e.g., the common pixel density).

Continuing the method 300, in the illustrative embodiment, the image comparison compute device 110 identifies one or more landmarks associated with the anatomical features (e.g., a subset of the anatomical features) to compare between the images (e.g., medical images) in the image data 130, as indicated in block 350 (which spans FIGS. 3D-3F). In doing so, as indicated in block 352, the image comparison compute device 110 may identify one or more landmarks to compare between images (e.g., medical images) from (e.g., produced by) different sources (e.g., the different imaging devices 140, 142, 144). The image comparison compute device 110 may identify one or more landmarks based on a predefined set (e.g., defined in a list or other data structure in the memory 214) of landmarks to be identified in the images, as indicated in block 354.

In some embodiments, the image comparison compute device 110 may identify the one or more landmarks based on (e.g., as a function of) a degree to which each landmark in a set of possible landmarks is presented in the images, as indicated in block 356. In doing so, the image comparison compute device 110 may identify the one or more landmarks based on a clarity of each landmark in the images, as indicated in block 358. As indicated in block 360, the image comparison compute device 110 may identify the landmarks based on a plane of the anatomy represented in each of the images. In other words, if a landmark in a set of possible landmarks for use is not visible from a particular angle or plane represented by the image, or is blurred, cropped out, or otherwise insufficiently represented, the image comparison compute device 110 may disregard that landmark in favor of one or more other landmarks in the set of possible landmarks for use. As indicated in block 362, the image comparison compute device 110 may identify one or more landmarks to be used by one or more machine learning models (e.g., the models 132) trained to compare image data. In doing so, as indicated in block 364, the image comparison compute device 110 may identify one or more landmarks based on a feature vector (e.g., a defined set of numeric input variables representing features of an object) associated with one or more convolutional neural networks (e.g., the models 132 may include one or more convolutional neural networks). For example, a model 132 may be configured to read a feature vector in which each of a set of elements in the vector represents a coordinate in two dimensions of a corresponding landmark.

The image comparison compute device 110 may identify (e.g., determine the location of) the sacral endplate of S1 (i.e., the endplate of the vertebra nearest the sacrum), as indicated in block 366. In some embodiments, the image comparison compute device 110 may determine the distance between opposite edges of the sacral endplate (e.g., the distance from the anterior (front) edge to the posterior (back) edge), as indicated in block 368. Additionally or alternatively, the image comparison compute device 110 may identify (e.g., determine the locations of) the anterior superior iliac spin (ASIS) and the pubic symphysis, as indicated in block 370. Further, as indicated in block 372, the image comparison compute device 110 may determine the distance between the ASIS and the pubic symphysis. Additionally or alternatively, the image comparison compute device 110 may identify the center of the femoral head and the midpoint of the sacral slope, as indicated in block 374. Further, the image comparison compute device 110 may determine the distance between the femoral head center and the sacral slope midpoint, as indicated in block 376. In the illustrative embodiment, in which a common scale (e.g., pixel density) has been established across the images, the image comparison compute device 110 may determine distances by defining a line between two points in each image and determining the number of pixels along the line.

Referring now to FIG. 3G, continuing the method 300, the image comparison compute device 110 may compare the one or more identified landmarks across the images to determine whether the images represent different individuals (or, by the same analysis, whether the images represent the same individual), as indicated in block 378. In doing so, as indicated in block 380, the image comparison compute device 110 may compare identified landmark(s) across images from different sources (e.g., from the X-ray imaging device 142 and the CT imaging device 144). As indicated in block 382, the image comparison compute device 110 may compare the identified landmark(s) across images from two dimensional image data (e.g., produced by the X-ray imaging device 142) and corresponding projections (e.g., planar projections) from three dimensional image data (e.g., produced by the CT imaging device 144). As indicated in block 384, in performing the comparisons, the image comparison compute device 110 may determine differences in locations of landmarks in the images. As indicated in block 386, the image comparison compute device 110 may determine differences in the distances between landmarks in the images (e.g., the distance between opposite edges of the sacral endplate, the distance between the ASIS and pubic symphysis, and/or the distance between the femoral head center and the sacral slope midpoint).

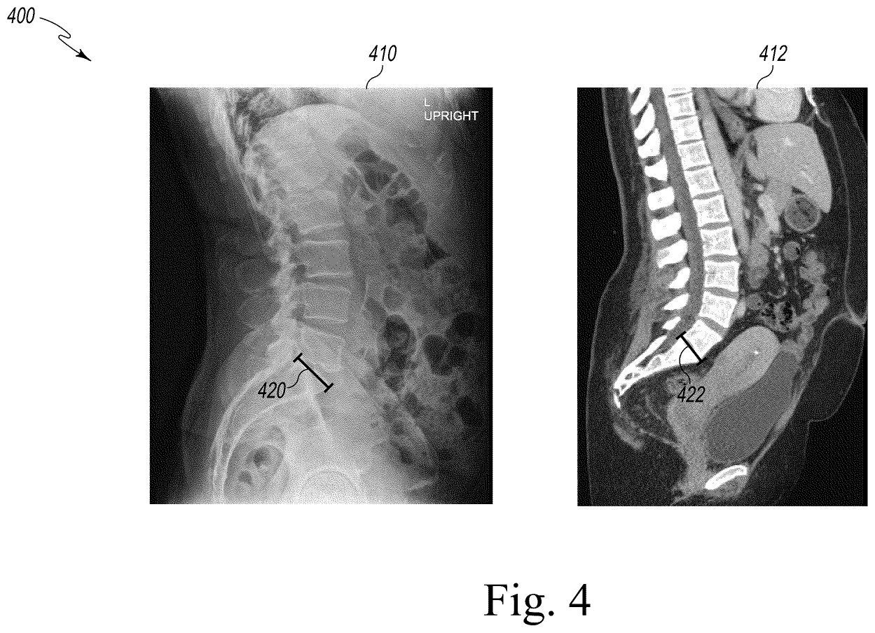

In block 388, the image comparison compute device 110 determines whether the differences satisfy a similarity threshold. As an example, referring briefly to FIG. 4, if the determined distance 420 between the edges of the sacral endplate in one image 410 differs from the determined distance 422 between the edges of the sacral endplate in another image 412 by a defined amount (e.g., 5%), the image comparison compute device 110 may determine that the images 410, 412 represent different individuals. In some embodiments, the similarity threshold may differ based on the comparison being performed (e.g., the distances between the ASIS and the pubic symphysis may differ by up to 7% before triggering a determination that the images represent different people). In some embodiments the similarity threshold is based on a score determined based on a combination of comparisons, which may be weighted according to their reliability in determining whether different individuals are represented across images.

Moving to FIG. 3H, in block 390, the image comparison compute device 110 determines the subsequent course of action based on whether the images have been determined to represent different individuals (in block 378). If the image comparison compute device 110 determined that different individuals are represented in the images, the method 300 advances to block 392 in which the image comparison compute device 110 produces a notification that the images have been determined to represent different individuals.

The image comparison compute device 110 may present the notification as a dialog box, a message, or other element in a user interface displayed by the image comparison compute device 110 (e.g., on a display device 224) and/or may send data and/or code (e.g., hypertext transfer markup language (HTML) code) to another compute device (e.g., the user compute device 150) indicative of the notification. In producing the notification, the image comparison compute device 110 may indicate (e.g., in the notification) a reason why the images were determined to represent different individuals, as indicated in block 394. For example, as indicated in block 396, the image comparison compute device 110 may indicate differences in the landmark(s) that did not satisfy the similarity threshold (e.g., from block 388). Referring back to block 390, in response to a determination that the images do not represent different individuals (i.e., that the images represent the same individual), the method 300 advances to block 398, in which the image comparison compute device 110 may produce a notification that the images have been determined to represent the same individual. In other embodiments, the image comparison compute device 110 may be configured to produce a notification only when the images have been determined to represent different individuals and may withhold any information regarding a comparison of the images if the images have been determined to represent the same individual.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A computer-implemented method for comparing medical images, the method comprising:
obtaining, with a compute device, image data produced by multiple imaging devices indicative of anatomical features associated with an arthroplasty procedure;
preprocessing, with the compute device, the image data, including projecting three dimensional image data produced by a first imaging device of the multiple imaging devices to a plane represented by two dimensional image data produced by a second imaging device of the multiple imaging devices;
identifying, with the compute device, one or more landmarks associated with the anatomical features for comparison between images in the image data;
comparing, with the compute device, the identified one or more landmarks across the images to determine whether the images represent different individuals; and
producing, with the compute device, a notification indicative of a result of the determination of whether the images represent different individuals.

2. The method of claim 1, wherein obtaining image data produced by multiple imaging devices comprises obtaining two dimensional X-ray image data produced by a fluoroscope and three dimensional computed tomography image data produced by a computed tomography imaging device.

3. The method of claim 1, wherein obtaining image data comprises obtaining preoperative, intraoperative, or postoperative image data of one or more joints.

4. The method of claim 3, wherein the one or more joints comprise a joint of a pelvis or a spine.

5. The method of claim 1, wherein projecting the three dimensional image data to a plane represented by the two dimensional image data comprises projecting the three dimensional image data to a sagittal plane, a coronal plane, a transverse plan, or an oblique plane represented in the two dimensional image data.

6. The method of claim 1, wherein preprocessing the image data comprises establishing a common scale among the images in the image data.

7. The method of claim 6, wherein establishing a common scale comprises establishing a common pixel density among the images in the image data.

8. The method of claim 1, wherein identifying one or more landmarks to compare comprises identifying one or more landmarks based on a predefined set of landmarks to be identified.

9. The method of claim 1, wherein identifying one or more landmarks to compare comprises identifying one or more landmarks to compare based on a degree to which each landmark in a set of possible landmarks is represented in the images.

10. The method of claim 9, wherein identifying one or more landmarks to compare based on a degree to which each landmark is represented in the images comprises identifying the one or more landmarks based on one or more of a clarity of each landmark in each of the images or an anatomical plane represented in each of the images.

11. The method of claim 1, wherein identifying one or more landmarks comprises identifying a sacral endplate.

12. The method of claim 11, further comprising determining, with the compute device, a distance between opposite edges of the sacral endplate.

13. The method of claim 1, wherein identifying one or more landmarks comprises identifying an anterior superior iliac spine (ASIS) and a pubic symphysis.

14. The method of claim 13, further comprising determining, by the compute device, a distance between the ASIS and the pubic symphysis.

15. The method of claim 1, wherein identifying one or more landmarks comprises identifying a femoral head center and a sacral slope midpoint.

16. The method of claim 15, further comprising determining, by the compute device, a distance between the femoral head center and the sacral slope midpoint.

17. The method of claim 1, wherein comparing the identified one or more landmarks across the images comprises determining differences in locations of the landmarks across the images.

18. The method of claim 1, wherein comparing the identified one or more landmarks across the images comprises determining differences in distances between the landmarks across the images.

19. The method of claim 1, wherein comparing the identified one or more landmarks across the images comprises determining whether differences in the landmarks across the images satisfy a similarity threshold.

20. The method of claim 1, wherein producing a notification comprises producing a notification indicative of differences in the one or more landmarks across the images in response to a determination that the images represent different individuals.
